(19) United States
(12) Patent Application Publication
(10) Pub. No.: US 2007/0072949 A1
(43) Pub. Date: Mar. 29, 2007

(54) Methods and apparatus for hydrogen gas production

(75) Inventors: James Anthony Ruud, Delmar, NY (US); Jennifer Lynn Molaison, Marietta, GA (US); Louis Andrew Schick, Delmar, NY (US); Anthony Yu-Chung Ku, Rexford, NY (US); Ke Liu, Rancho Santa Margrita, CA (US); Parag Prakash Kulkarni, Tustin, CA (US); R. George Rizeq, Mission Viejo, CA (US)

Correspondence Address: JOHN S. BEULICK;(12764), ARMSTRONG TEASDALE LLP, ONE METROPOLITAN SQUARE, SUITE 2600, ST. LOUIS, MI, 63102-2740, US

(73) Assignee: General Electric Company

(21) Appl. No.: 11/263,269

(22) Filed: Oct. 31, 2005

Related U.S. Application Data
(60) Provisional application No. 60/721,560, filed on Sep. 28, 2005.

(51) Int. Cl. C07C 27/06; F02C 3/30

(52) U.S. Cl. 518/702; 60/775

(57) ABSTRACT

An apparatus for producing hydrogen gas, wherein the apparatus includes a reactor. The reactor includes a catalyst, a membrane in flow communication with the catalyst, and a heat exchanger integrated with the reactor.

### CROSS REFERENCE TO RELATED APPLICATIONS

This application is a non-provisional of and claims priority from U.S. Provisional Patent Application Ser. No. 60/721,560, filed on Sep. 28, 2005, and is related to co-pending U.S. Patent Application entitled: FUNCTIONALIZED INORGANIC MEMBRANES FOR GAS SEPARATION, (Atty Dkt. No.: 162652/2) the entire contents of both are hereby incorporated by reference in their entirety.

### STATEMENT REGARDING FEDERALLY SPONSORED RESEARCH & DEVELOPMENT

This invention was made with Government support under contract number DOE NETL DE-FC26-05NT42451 awarded by the U.S. Department of Energy. The Government may have certain rights in the invention.

### BACKGROUND OF THE INVENTION

This invention relates generally to gas separation processes, and more particularly, to syngas conversion and purification for hydrogen production.

The application of syngas conversion and purification after a coal gasifier can be used for integrated gasification combined cycle (IGCC) power plants for electricity production from coal. It can also be used for IGCC-based polygeneration plants that produce multiple products such as hydrogen and electricity from coal, and it is usefull for plants that include carbon dioxide separation. It is also applicable to purification of other hydrocarbon-derived syngas which can be used for electricity production or polygeneration, including syngas derived from natural gas, heavy oil, biomass and other sulfur-containing heavy carbon fuels.

The commercialization of known ‘coal to-hydrogen (H₂) and electricity’ technologies (IGCC power plants or coal gasification-based polygeneration plants) has been hampered by the high capital costs associated with removing the most significant impurities, such as sulfur, present in coal. The stringent purity requirements for hydrogen fuel and the fuel specifications for the gas turbine are generally satisfied using a series of clean-up unit operations, which facilitate carbon monoxide (CO) conversion, sulfur removal, carbon dioxide (CO₂) removal and final gas polishing. The syngas produced can be sent to a combined cycle plant to produce electricity. Since syngas is a feedstock for manufacturing chemical and fuels, it can also be used in a polygeneration plant that integrates a combined cycle power plant and chemical reactors for polygeneration of electricity and chemical products. The chemical products can include hydrogen, ammonia, methanol, dimethyl ether and Fischer-Tropsch gasoline and diesel fuels. The CO₂ rich stream can be compressed and sent to sequestration.

Some known syngas clean-up technologies focus on removing each impurity in a separate unit operation. Raw fuel gas exiting the gasifier is cooled and cleaned of particulate before being routed to a series of sulfur removal units and water-gas-shift (WGS) reactors. Those unit operations convert CO and H₂O present in the syngas to CO₂ and H₂, thereby concentrating it in the high-pressure raw fuel gas stream. Once concentrated, CO₂ and sulfur present in the stream can be removed using low temperature amine-based absorption processes. CO₂ is then dried and compressed to supercritical conditions for pipeline transport. Part of the clean fuel gas from the amine-based unit, now rich in H₂, is either fired directly in a combustion turbine, or used in other polygeneration systems. Waste heat is recovered from the process and used to raise steam to feed to a steam turbine. Part of the clean stream can purified further to produce fuel grade H₂ product. However, because of the different operating requirements and parameters of each unit, known clean-up technologies may be expensive. Moreover, because of the large number of unit operations used, known clean-up technologies generally require large footprints within a plant. For example, at least some known units have auxiliary requirements for solvent regeneration and pollutant recovery. Known units involve low temperature processes that require the gas stream to be cooled resulting into energy loss and lower efficiency.

### BRIEF DESCRIPTION OF THE INVENTION

In one aspect, an apparatus for producing hydrogen gas is provided. The apparatus includes a reactor, wherein the reactor includes a catalyst and a membrane in flow communication with the catalyst. The reactor also includes a heat exchanger integrated with the reactor.

In another aspect, a method for separating hydrogen from a fuel source is provided. The method includes forming a first gaseous fuel mixture from a gasification process and forcing the first gaseous fuel mixture through a water-gas-shift reactor including a carbon dioxide and hydrogen sulfide selective membrane in flow communication with a catalyst, wherein the catalyst is cooled by a heat exchanger. The method also includes forming a second gaseous fuel mixture, wherein the second gaseous mixture includes more hydrogen than the first gaseous fuel mixture. The method further includes removing at least one of carbon dioxide and hydrogen sulfide from the second gaseous fuel mixture.

In a further aspect, a plant is provided. The plant includes a gasification unit coupled to a carbonyl sulfide hydrolysis unit to produce a fuel gas mixture and a water-gas-shift reactor configured to produce hydrogen and carbon dioxide. The reactor includes a catalyst, a high-temperature, carbon dioxide and hydrogen sulfide selective membrane in flow communication with the catalyst, and a heat exchanger integrated with said reactor. The plant also includes a combined cycle power generation unit configured to produce electricity.

### BRIEF DESCRIPTION OF THE DRAWINGS

FIG. 1 is a schematic view of an exemplary integrated gasification combined cycle (IGCC) polygeneration plant including a known syngas clean-up section.

FIG. 2 is a schematic view of an exemplary embodiment of a IGCC polygeneration plant including an integrated syngas clean-up section.

### DETAILED DESCRIPTION OF THE INVENTION

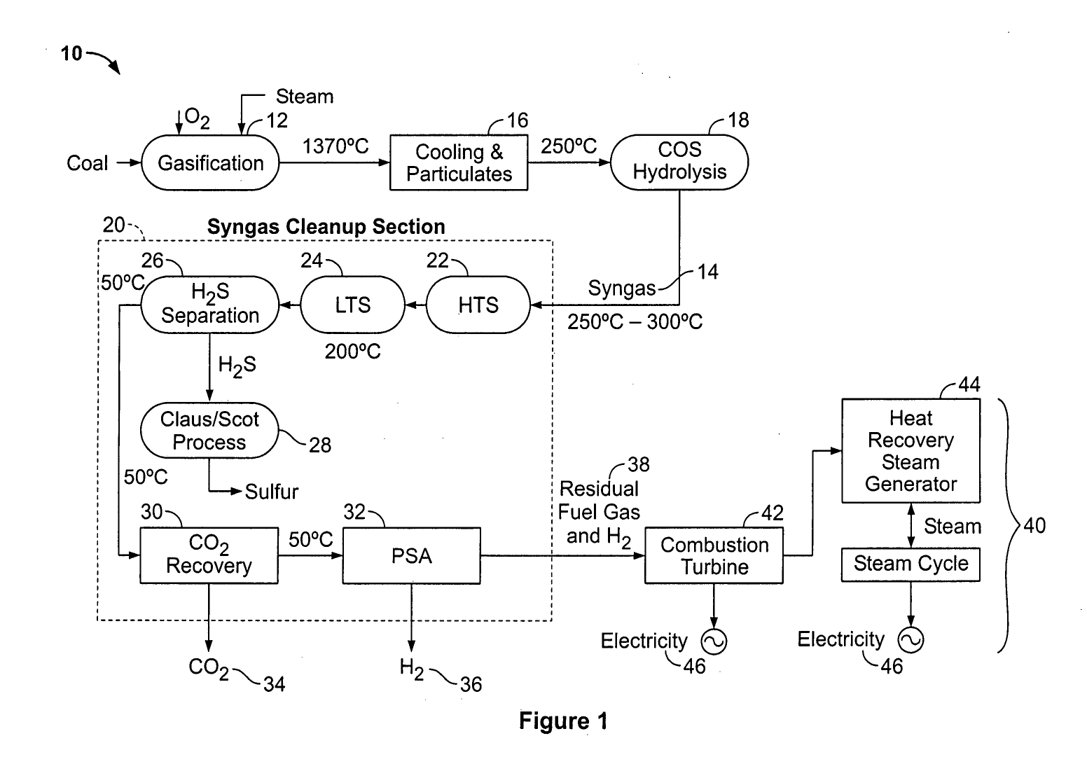

FIG. 1 is a schematic view of an exemplary integrated gasification combined cycle (IGCC) polygeneration plant 10 for hydrogen gas (H₂) and electricity production with carbon dioxide (CO₂) separation. Plant 10 includes a gasification unit 12 that receives coal, oxygen containing material, and high temperature steam or water therein and produces a syngas 14. Gasification unit 12 is in flow communication with a series of syngas coolers 16 configured to remove heat and particulates and with a carbonyl sulfide (COS) hydrolysis unit 18 that is configured to convert COS to hydrogen sulfide (H₂S) in the syngas 14. Syngas 14 is then processed through a known syngas clean-up section 20. In the exemplary embodiment, clean-up section 20 includes six individual unit operations including a high-temperature shift (HTS) reactor 22, a low temperature shift (LTS) reactor 24, a H₂S separation unit 26, a solvent regeneration (Claus/Scot processes) unit 28, a CO₂ recovery unit 30, and pressure swing adsorption (PSA) unit 32. HTS 22 includes a catalyst optimized for high temperature (about 300-400° C.) operation and LTS 24 includes a catalyst optimized for low temperature (about 200° C.) operation.

During operation, a thermodynamically limited water-gas-shift reaction (CO+H₂O⇄CO₂+H₂) converts carbon monoxide (CO) to CO₂, but does not proceed to completion in the presence of CO₂, thus leaving approximately 1% CO in syngas 14. Syngas 14 is then cooled to approximately 50° C. such that, the majority of steam present in syngas 14 is condensed, along with any water-soluble acid gases such as, but not limited to, hydrogen chloride (HCl) and/or ammonia (NH₃). H₂S is then typically removed using either a physical or a chemical absorption process in H₂S separation unit 26. Both H₂S removal processes require the use of solvents, which are regenerated in solvent regeneration unit 28 and elemental sulfur (S) is produced. Gas exiting H₂S separation unit 26 enters CO₂ recovery unit 30 wherein the CO₂ 34 is removed by using a solvent similar to one used in H₂S separation unit 26. After CO₂ recovery, syngas 14 enters PSA 32, which facilitates removing any remaining impurities, providing approximately 99.99% pure H₂ 36. PSA 32 also provides residual fuel gas and H₂ 38, which are in turn used by a combined cycle power generation unit 40 which includes a combustion turbine 42 and a heat recovery steam generator 44 to produce electricity 46.

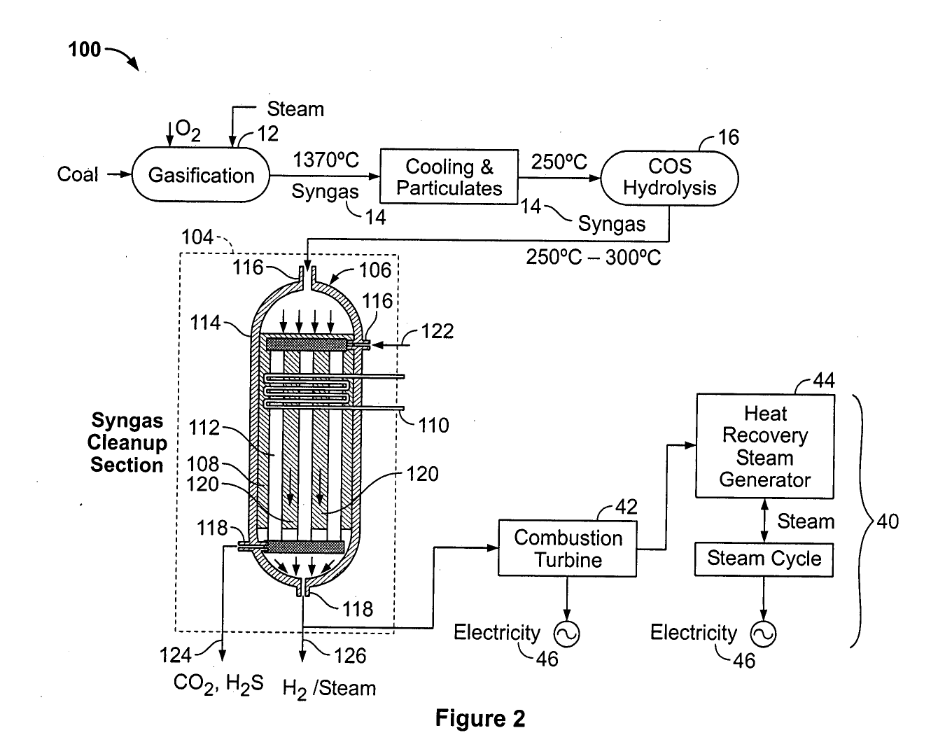

FIG. 2 is an exemplary embodiment of a IGCC polygeneration plant 100 for H₂ and electricity production with CO₂ separation. IGCC plant 100 is similar to IGCC plant 10, (shown in FIG. 1) and components of IGCC plant 100 that are identical to IGCC plant 10 are identified in FIG. 2 using the same reference numbers used in FIG. 1.

In the exemplary embodiment, IGCC plant 100 is configured to process syngas 14 through an exemplary embodiment of an integrated, high temperature syngas clean-up section 104. Integrated section 104 combines a six-step, capital-intensive process series into a single, simplified operation. Specifically, integrated section 104 includes a water-gas-shift reactor 106 that includes a shift reaction catalyst 108, an active cooling heat exchanger 110, and a high-temperature membrane 112. The integrated section 104 allows for a water-gas shift reaction and CO₂ separation to occur within reactor 106.

In the exemplary embodiment, reactor 106 comprises a shell 114 including a plurality of input channels 116 and a plurality of output channels 118. Reactor 106 is configured to receive syngas 14 through a first input channel 116. Syngas 14 enters reactor 106 having a temperature approximately between 250° C. and 300° C.

In the exemplary embodiment, shift reactor catalyst 108 is configured to convert CO to CO₂. In one embodiment, shift reactor catalyst 108 includes Iron (Fe) and Ferro chromium (Fe—Cr) alloys. In another embodiment, shift reactor catalyst 108 is a noble metal catalyst such as, but not limited to, Palladium (Pd), Platinum (Pt), Rhodium (Rh), or Platinum rhenium (Pt—Re) supported on high surface area ceramics such as, but not limited to, Cerium oxide (CeO₂) or Aluminum Oxide (Al₂O₃). In the exemplary embodiment, catalyst 108 is packed within shell 114 such that heat exchanger 110 and membrane 112 are substantially encapsulated within catalyst 108.

As syngas 14 travels through catalyst 108 within shell 114, an exothermic water-gas shift reaction (CO+H₂O⇄CO₂+H₂) converts CO to CO₂. Heat exchanger 110 facilitates removing excess heat from the exothermic shift reactions by actively cooling catalyst 108. Catalyst 108, heat exchanger 110, membrane 112 consolidate two unit operations, HTS 20 and LTS 22 (shown in FIG. 1) into one operation within reactor 106.

In the exemplary embodiment, membrane 112 is CO₂ selective and thus continuously removes the CO₂ produced in the water-gas-shift reactor 106, allowing the equilibrium conversion of CO to CO₂ to proceed to nearly complete CO removal (approximately 10 ppm CO in H₂ product). Membrane 112 is substantially encapsulated within catalyst 108 such that CO₂ produced in the water-gas-shift reaction is removed from H₂ stream 126. Membrane 112 is also H₂S selective and thus continuously removes H₂S to facilitate achieving low levels of H₂S (<100 ppb) in the H₂ product. Furthermore, membrane 112 is operable at a high temperature. For example, in the exemplary embodiment, membrane 112 is operable at an increased temperature i.e., between approximately 250-500° C. this is a temperature increase from 50° C. to greater than 250° C. as compared to FIG. 1. The increased operating temperature facilitates reducing energy losses associated with cooling and reheating. Integrated section 104 operates at temperatures between approximately 250° and 500° C. Suitable membranes are describe in U.S. Patent Application entitled: FUNCTIONALIZED INORGANIC MEMBRANES FOR GAS SEPARATION, (Atty. Dkt. No.: 162652/2).

During operations, in the exemplary embodiment, CO₂ and H₂S pass through membrane 112 to a plurality of center of the membrane tubes 120. A low quality steam or a sweep gas 122 is introduced to reactor 106 through a second input channel 116 to remove CO₂ and H₂S from reactor 106 through a first output channel 118 in a first separate stream 124 which is enriched in CO₂ and H₂S. The bulk of processed syngas 14 exits reactor 106 through a second output channel 118 in a second stream 126 of steam and H₂, which is depleted in CO₂ and H₂S. In alternative embodiments, CO₂ passes through a first CO₂-selective membrane 112, wherein a first sweep gas 122 is introduced to remove CO₂ from reactor 106 into a CO₂ enriched stream, and H₂S passes through a second H₂S-selective membrane 112, wherein a second sweep gas 122 is introduced to remove H₂S from reactor 106 into a H₂S-enriched stream, and the bulk of processed syngas 14 exits as a third, H₂ containing stream, which is depleted in CO₂ and H₂S.

In another embodiment, membrane 112 can be constructed from two separate materials, wherein the first material is selective for CO₂ and the second is selective for H₂S. In this embodiment, the CO₂ selective membrane is substantially encapsulated within catalyst 108. The H₂S-selective membrane can be located downstream of catalyst 108 in the path of the water-gas-shift product gas. The result is three separate streams exiting reactor 106, the first stream for H₂, the second for CO₂, and the third for H₂S. The third stream can be further converted to elemental sulfur or sulfuric acid.

The above-described reactor system based on high-temperature membrane separation of carbon dioxide from syngas offers many advantages for an integrated coal-to-H₂ and electricity polygeneration process. The integrated concept allows for a reduced energy cost for CO₂ capture, lower capital cost, and a smaller overall footprint for the plant. Furthermore, the integrated approach leverages synergies between water-gas shift reactions and the need for CO₂ removal. The use of membranes for H₂S removal eliminates the need for energy-intensive solvent regeneration and sulfur recovery units. The economic benefits of the module will facilitate commercialization of IGCC electricity generation plants or IGCC polygeneration with CO₂ separation plants. The elimination of four unit operations (H₂S removal, CO₂ removal, solvent regeneration and PSA) and the consolidation of two others (HTS, LTS) into an integrated module will significantly reduce capital costs which will have a significant impact on the economic feasibility of coal-based H₂ production technologies.

An exemplary embodiment of an integrated, high temperature syngas clean-up section is described in detail above. The syngas clean-up section is not limited to the specific embodiments described herein, but rather, components of the clean-up section may be utilized independently and separately from other components described herein. Furthermore, the need to remove CO₂ is not unique to coal-derived plants, and as such, the integrated syngas clean-up section could be used for alternative fuel or biomass systems to convert low-value syngas to high-purity H₂. Therefore, the present invention can be implemented and utilized in connection with many other fuel systems and turbine configurations.

While the invention has been described in terms of various specific embodiments, those skilled in the art will recognize that the invention can be practiced with modification within the spirit and scope of the claims.

What is claimed is:

1. An apparatus for producing hydrogen gas, said apparatus comprising a reactor, said reactor comprising:
a catalyst;
a membrane in flow communication with said catalyst; and
a heat exchanger integrated with said reactor.

2. An apparatus in accordance with claim 1 wherein said reactor is a water-gas shift reactor configured to receive a syngas and to produce hydrogen gas, said reactor comprises a shell comprising a plurality of input channels and a plurality of output channels, said shell configured to contain an exothermic water-gas shift reaction.

3. An apparatus in accordance with claim 1 wherein said catalyst comprises a packed bed of shift catalyst configured to convert carbon monoxide and steam to carbon dioxide and hydrogen.

4. An apparatus in accordance with claim 1 wherein said heat exchanger is configured to remove excess heat from exothermic shift reactions by active cooling of said reactor.

5. An apparatus in accordance with claim 1 wherein said membrane comprises at least one of a high-temperature carbon dioxide and hydrogen sulfide selective membrane configured to selectively remove at least one of carbon dioxide and hydrogen sulfide.

6. A method for separating hydrogen from a fuel source, said method comprising:
forming a first gaseous fuel mixture from a gasification process;
forcing the first gaseous fuel mixture through a water-gas-shift reactor including a carbon dioxide and hydrogen sulfide selective membrane in flow communication with a catalyst, wherein the reactor is cooled by a heat exchanger;
forming a second gaseous fuel mixture, wherein the second gaseous mixture comprises more hydrogen than the first gaseous fuel mixture; and
removing at least one of carbon dioxide and hydrogen sulfide from the second gaseous fuel mixture.

7. A method in accordance with claim 6 wherein the fuel source is selected from at least one of a coal, a natural gas, an oil, and a biomass.

8. A method in accordance with claim 6 wherein in the reactor maintains a temperature between approximately 250° C. and 500° C.

9. A method in accordance with claim 6 wherein the catalyst is a material configured to initiate an exothermic water-gas-shift reaction such that the fuel gas mixture is converted to carbon dioxide and hydrogen gas.

10. A method in accordance with claim 6 wherein the heat exchanger is configured to actively cool the reactor.

11. A method in accordance with claim 6 wherein the membrane comprises at least one of a high-temperature carbon dioxide and hydrogen sulfide selective membrane configured to selectively remove at least one of carbon dioxide and hydrogen sulfide.

12. A method in accordance with claim 6 wherein removing at least one of carbon dioxide and hydrogen sulfide from the second gaseous fuel mixture further comprises removing carbon dioxide and hydrogen sulfide into a first stream by introducing at least one of a steam or a sweep gas into the membrane.

13. A method in accordance with claim 6 wherein removing at least one of carbon dioxide and hydrogen sulfide from the second gaseous fuel mixture further comprises removing carbon dioxide into a first stream and removing hydrogen sulfide into a second stream by introducing at least one of a steam or a sweep gas into the membrane.

14. A plant comprising:
a gasification unit coupled to a carbonyl sulfide hydrolysis unit to produce a fuel gas mixture;
a water-gas-shift reactor configured to produce hydrogen and carbon dioxide, said reactor comprising:
a catalyst;
a high-temperature, carbon dioxide and hydrogen sulfide selective membrane in flow communication with said catalyst;
a heat exchanger integrated with said reactor; and
a combined cycle power generation unit configured to produce electricity.

15. A plant in accordance with claim 14 wherein said plant is at least one of a integrated gas turbine/steam turbine combined cycle plant and a coal gasification-based polygeneration plant.

16. A plant in accordance with claim 14 wherein said catalyst is a water-gas-shift reactor material configured to initiate an exothermic water-gas-shift reaction such that said fuel gas mixture is converted to carbon dioxide and hydrogen gas.

17. A plant in accordance with claim 14 wherein said membrane comprises at least one of a high-temperature carbon dioxide and hydrogen sulfide selective membrane configured to selectively remove at least one of carbon dioxide and hydrogen sulfide.

18. A plant in accordance with claim 14 wherein said reactor is configured to introduce at least one of a steam or a sweep gas into said membrane such that hydrogen exits said reactor as a first stream and carbon dioxide and hydrogen sulfide exit said reactor as a second stream.

19. A plant in accordance with claim 14 wherein said reactor is configured to introduce at least one of a steam or a sweep gas into said membrane such that hydrogen exits said reactor as a first stream, carbon dioxide exits said reactor as a second stream, and hydrogen sulfide exits said reactor as a third stream.

20. A plant in accordance with claim 14 wherein said reactor maintains a temperature between approximately 250° C. and 500° C.
